(19) United States
(12) Patent Application Publication
(10) Pub. No.: US 2009/0088651 A1
(43) Pub. Date: Apr. 2, 2009

(54) Method and apparatus to perform transvascular hemodynamic sensing

(76) Inventors: Allan Charles Shuros, St. Paul, MN (US); Michael J. Kane, Lake Elmo, MN (US)

Correspondence Address: HOLLINGSWORTH & FUNK, LLC, 8009 34TH AVE S., SUITE 125, MINNEAPOLIS, MN, 55425, US

(21) Appl. No.: 11/904,946

(22) Filed: Sep. 28, 2007

(51) Int. Cl. A61B 5/0215

(52) U.S. Cl. 600/488

(57) ABSTRACT

An implantable transvascular pressure sensing device may include at least one deflection sensor configured to output a deflection signal indicative of vascular wall deflections. A implantable transvascular pressure sensing device may further include an implantable sensor support member attached to the deflection sensor, the sensor support member configured to facilitate contact between the deflection sensor and a first vessel wall at a portion of a first vessel proximate a second vessel, wherein the portion of the first vessel wall is adjacent to the second vessel such that wall deflections of the second vessel deflect the portion of the first vessel wall. A method of measuring a parameter of a vessel can include generating a deflection signal from a sensor within a vein, the deflection signal indicative of venous wall deflections of the vein, and determining a parameter value associated with a vessel contacting the vein using the deflection signal.

### FIELD OF THE INVENTION

The present invention relates generally to monitoring and therapy devices and methods, and, more particularly, to monitoring hemodynamic parameters using an implantable device.

### BACKGROUND OF THE INVENTION

When functioning normally, the heart produces rhythmic contractions and is capable of pumping blood throughout the body. The heart has specialized conduction pathways in both the atria and the ventricles that enable excitation impulses (i.e. depolarizations) initiated from the sino-atrial (SA) node to be rapidly conducted throughout the myocardium. These specialized conduction pathways conduct the depolarizations from the SA node to the atrial myocardium, to the atrio-ventricular node, and to the ventricular myocardium to produce a coordinated contraction of both atria and both ventricles.

The conduction pathways synchronize the contractions of the muscle fibers of each chamber as well as the contraction of each atrium or ventricle with the opposite atrium or ventricle. Without the synchronization afforded by the normally functioning specialized conduction pathways, the heart's pumping efficiency is greatly diminished. Patients who exhibit pathology of these conduction pathways can suffer compromised cardiac output.

Cardiac rhythm management (CRM) devices have been developed that provide pacing stimulation to one or more heart chambers in an attempt to improve the rhythm and coordination of atrial and/or ventricular contractions. CRM devices typically include circuitry to sense signals from the heart and a pulse generator for providing electrical stimulation to the heart. Leads extending into the patient's heart chamber and/or into veins of the heart are coupled to electrodes that sense the heart's electrical signals and deliver stimulation to the heart in accordance with various therapies for treating cardiac arrhythmias and dyssynchrony.

CRM devices commonly need to sense various cardiac parameters to diagnose cardiac conditions and determine an appropriate therapy. Many of the arrhythmia discrimination methods currently used rely on electrocardiograms (ECG) based techniques.

Monitoring of cardiac function using ECG signals is subject to error caused by several factors, including the presence of other electrical signals (noise) in the body and blanking periods during therapy. Moreover, sensed electrocardiac signals may not be indicative of actual heart output. For example, ECG methodologies may be able to diagnose a fibrillation condition, but can have difficulty estimating actual cardiac output during fibrillation.

Therefore, despite the utility of EGM-based methods employed in implantable cardiac devices, these methods may be susceptible to error and are not suitable in all conditions for characterizing cardiac output.

The present invention provides methods and systems for performing transvascular hemodynamic sensing and provides various advantages over the prior art.

### SUMMARY OF THE INVENTION

The present invention involves approaches for performing transvascular hemodynamic sensing. One embodiment of the invention is directed to an implantable transvascular pressure sensing device that includes at least one deflection sensor configured to output a deflection signal indicative of vascular wall deflections and an implantable sensor support member attached to the deflection sensor, the sensor support member configured to facilitate contact between the deflection sensor and a first vessel wall at a portion of a first vessel proximate a second vessel, wherein the portion of the first vessel wall is adjacent to the second vessel such that wall deflections of the second vessel deflect the portion of the first vessel wall.

A method for performing transvascular hemodynamic sensing can include generating a deflection signal from a sensor within a vein, the deflection signal indicative of venous wall deflections of the vein, and determining a parameter value associated with a vessel contacting the vein using the deflection signal. In some embodiments of the present invention, the parameter value is a heart rate.

The method for performing transvascular hemodynamic sensing can further include generating a reference signal from a reference sensor located within the vein but spaced apart from the sensor, the reference signal indicative of venous wall deflection of the vein, and determining the parameter value using the deflection signal and the reference signal. In some embodiments of the present invention, the parameter value can be arterial blood pressure.

One embodiment of the invention includes means for generating a deflection signal from a sensor within a vein, the deflection signal indicative of venous wall deflections of the vein, and means for determining a parameter value associated with a vessel contacting the vein using the deflection signal.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a diagram illustrating a vessel crossover junction, which can be used in accordance with embodiments of the invention;

FIG. 2 is a diagram illustrating a sensing device for performing transvascular hemodynamic sensing using, for example, the vessels of FIG. 1, in accordance with embodiments of the invention;

FIGS. 3A and 3B are diagrams illustrating different views of a lead based sensing device for performing transvascular hemodynamic sensing of parallel vessels in accordance with embodiments of the invention;

FIG. 4 is a diagram illustrating a lead based sensing device for performing transvascular hemodynamic sensing of perpendicular vessels in accordance with embodiments of the invention;

FIG. 5 is a diagram illustrating a helix based sensing device for performing transvascular hemodynamic sensing of parallel vessels in accordance with embodiments of the invention;

FIG. 6 is a diagram illustrating a stent based sensing device for performing transvascular hemodynamic sensing of parallel vessels in accordance with embodiments of the invention;

FIG. 7 is a diagram illustrating an ultrasonic based sensing device for performing transvascular hemodynamic sensing of crossing vessels in accordance with embodiments of the invention;

FIG. 8 is a diagram illustrating an implanted therapy device with leads which can be used for performing transvascular hemodynamic sensing in accordance with embodiments of the invention;

FIG. 9 is a therapy device incorporating circuitry capable of implementing transvascular hemodynamic sensing in accordance with embodiments of the invention;

FIG. 10 is a flowchart illustrating a method of performing transvascular hemodynamic sensing in accordance with embodiments of the invention; and

FIG. 11 is a flowchart illustrating a method of performing transvascular hemodynamic sensing in accordance with embodiments of the invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings forming a part hereof, and in which are shown by way of illustration, various embodiments by which the invention may be practiced. It is to be understood that other embodiments may be utilized, and structural and functional changes may be made, without departing from the scope of the present invention.

Systems, devices or methods according to the present invention may include one or more of the features, structures, methods, or combinations thereof described herein. For example, a device or system may be implemented to include one or more of the advantageous features and/or processes described below. It is intended that such a device or system need not include all of the features described herein, but may be implemented to include selected features that provide for useful structures and/or functionality. Such a device or system may be implemented to provide a variety of therapeutic or diagnostic functions.

Blood pressure, heart rate parameters, and other hemodynamic parameters can be used to diagnose and monitor physiologic conditions and assist in delivering and evaluating therapies. For example, a rapid heart rate can be associated with tachyarrhythmia, a condition characterized by rapid and disorganized contraction of one or more heart chambers. Tachyarrhythmia can be a precursor to fibrillation, a potentially fatal heart condition. Therapies exist for both tachyarrhythmia and fibrillation, including electrical stimulation therapies deliverable by implantable medical devices.

Despite the existence of therapies to treat heart conditions, the particular heart conditions should first be accurately detected in order to ensure the delivery of an appropriate therapy.

Cardiac electrical signals are widely used to determine heart rate and other cardiac parameters to diagnose and monitor cardiac conditions. Cardiac electrical signals may be sensed via surface electrodes or implantable signals to generate an electrocardiogram (ECG) or electrogram (EGM) signal. However, monitoring cardiac electrical signals using ECG or EGM signals are subject to error caused by several factors. One such factor is that the body's non-cardiac muscles, such as skeletal muscles, also produce electrical signals which appear as noise on an ECG or EGM. Also, blanking periods for sensing cardiac signals exist during and shortly after delivery of electrical therapies, as the electrical stimulation would otherwise appear as large magnitude signals on an ECG or EGM, obscuring lower magnitude cardiac signals.

Analysis of cardiac electrical activity also has limited capability to determine actual cardiac output, particularly in tachyarrhythmia and fibrillation conditions, as well as when electrical therapy is being delivered, as discussed above. Therefore, despite the utility of EGM-based methods employed in implantable cardiac devices, these methods may be susceptible to error and are not suitable in all conditions.

Hemodynamic parameters can alternatively be mechanically measured. The heart expels blood into the arterial system. Contraction of heart chambers, the left ventricle in particular, can produce waves of blood flowing through the arterial system. Many arteries exhibit elastic properties, such that an increase in pressure associated with a ventricular contraction will circumferentially distend an artery. Therefore, each left ventricular contraction can be associated with a momentary increase in blood pressure and/or distension of an artery as the wave of blood travels through an artery.

The presence of cardiac pulse, or heartbeat, has previously been detected by palpating the patient's neck and sensing changes (physical expansion and contraction) in the volume of the patient's carotid artery due to blood pumped from the patient's heart. When the heart's ventricles contract during a heartbeat, a pressure wave is sent throughout the patient's peripheral circulation system. Arterial blood pressure typically rises with the ventricular ejection of blood during systole, appearing as consecutive blood pressure waves in the arterial system. Arterial blood pressure falls off again as the ventricles expand to fill with blood from the aortas (diastolic state).

Traditional external measuring of hemodynamic parameters, such as depressing the skin by hand along the neck or wrist to sense systolic blood pulses, is subject to inaccuracies inherent in sensing though the skin and other tissues. Additionally, such methods of hemodynamic parameter monitoring are not practicable in the field of implantable monitoring and therapeutic devices, which offer superior monitoring and therapeutic convenience and automaticity.

Monitoring hemodynamic parameters in arteries, including blood pressure and pulse rate, can be of particular importance. Mechanical sensing of arterial blood parameters are less susceptible to interference by pacing and defibrillation pulses, as compared to ECG/EGM based methods, and can monitor parameters, such as blood pressure, that ECG/EGM based methods cannot.

Direct measuring of hemodynamic parameters can be performed by a mechanical sensor placed within an artery. However, sensing blood pressure and/or arterial pulses with a sensor placed within an artery presents particular problems. For example, the relatively high pressures and flow rates of blood within arteries can lead to clot formation on the sensor and/or support member. Clot formation, known as thrombosis, can break free and drift within the artery as an embolus. Emboli can lodge within arteries and restrict blood flow, raising the risks of tissue damage and potentially fatal heart attacks and strokes.

Hemodynamic parameters of the venous system can be inadequate to detect and diagnose cardiac conditions. Systolic blood pulses are experienced less dramatically in the venous system, as compared to the arterial system, because the left ventricle pumps blood directly into the arterial system. Therefore, monitoring of arterial blood pressure can provide more information regarding heart rate, maximum heart ejection pressure, output volume, and other hemodynamic parameters, as compared to monitoring of the venous pressure.

Devices of the present invention provide for implantable sensing of arterial blood parameters without entering an artery. Wall deflections of the artery can be measured via a vessel adjacent to the artery. The arterial wall deflections can be used to detect systolic blood pulses and measure various parameters of the cardiac output and arterial blood flow. Because devices and methods of the present invention measure wall deflections of a vessel adjacent to the artery, blood parameter measurement can be conducted without invasively disturbing the vessel of interest, such as a high pressure artery. Moreover, measurements according to the present invention would not be subject to electrical noise and blanking periods that complicates ECG/EGM based methods, as well as offer the ability to monitor parameters that ECG/EGM methods cannot sense.

Vessels, including various veins and arteries, are often located adjacent to one another within the body. Adjacent vessel sections may run parallel or cross over one another. These proximate vessels can be made to contact one another, such that a relatively high pressure blood pulse traveling through one vessel may temporarily deflect the circumference of a portion of the vessel, causing a deflection in a wall of the adjacent vessel. Sensing the displacement and/or pulsatile movement of the adjacent vessel wall induced by the blood pulse allows monitoring of the hemodynamic status of the vessel of interest without entering that vessel.

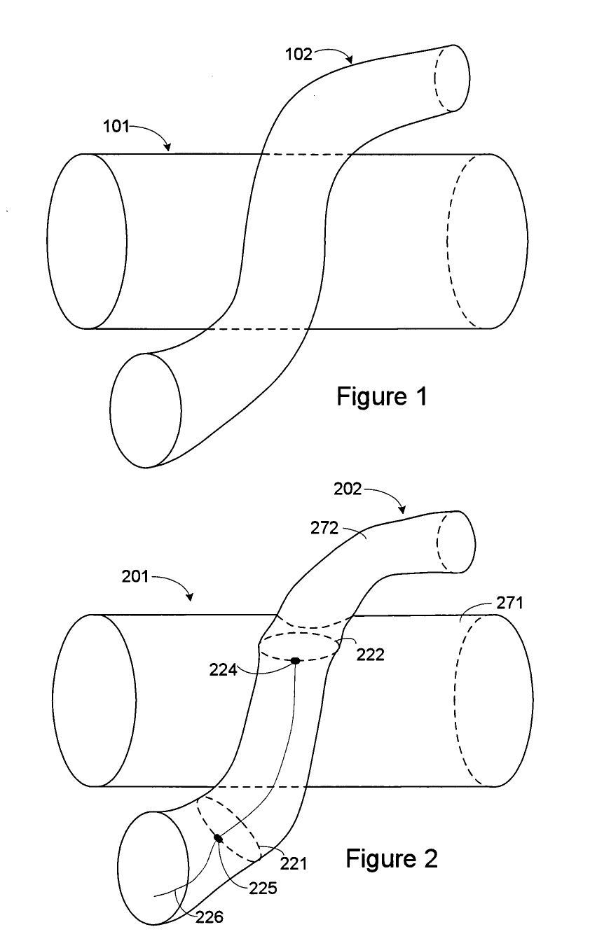

FIG. 1 illustrates a vessel crossover. A vein 102 is illustrated crossing over the left circumflex coronary artery 101 (LCX). The LCX is located proximate the heart and receives blood expelled from the left ventricle into the coronary artery system. Therefore, the LCX 101 is sensitive to systolic blood pulses, each of which can cause circumferential distention of the LCX 101 walls.

Vein 102 is proximate the LCX 101. When contacting the LCX 101, the vein 102 may experience wall deflections as systolic blood pulses cause circumferential distention of the LCX 101.

There are many bodily vessel combinations that could be used with apparatuses and methods of the present invention. Such vessel combinations include, but are not limited to, the azygous vein adjacent to the aorta, the subclavian vein adjacent to the subclavian artery, the superior vena cava adjacent to aorta and/or brachiocephalic artery or right subclavian artery, and internal/external jugular veins adjacent carotid arteries, as well as any other vessel combinations in the body where blood flow dynamics in one vessel cause a vessel wall deflection in an adjacent vessel (even if such vessel wall deflection can only be sensed by biasing the vessel).

FIG. 2 illustrates a vessel crossover, which can represent the LCX/vein crossover discussed above. Continuing with the example discussed above in association with FIG. 1, a measuring device has been provided within the vein 202. The measuring device comprises sensor 224, ring 222, and lead 226. Optionally, the measuring device also includes reference sensor 225 and ring 221.

The sensors 224 and 225 are respectively attached to rings 221 and 222. The rings 221 and 222 serve as support structures for sensors 224 and 225, respectively, anchoring the sensors 224 and 225 and placing them into contact with the vein 202 walls. Ring 222 may also serve to force contact between the vein 202 and LCX 201 by diametrically enlarging a portion of the vein 202, such that expansion of the vein 202 diameter overcomes any natural distance between the vein 202 and LCX 201 outer wall surfaces.

Sensor 224 is anchored to the vein wall 272 at a location where a portion of the exterior surface of the vein wall 272 is in contact with a portion of the exterior surface of the artery wall 271. The sensors 224 and 225 can detect and measure wall detections of the vein 202 at, and/or near, the areas of the vein wall 272 that the sensors 224 and 225 are respectively anchored. Therefore, sensor 224 can detect deflections of the artery wall 271 caused by systolic blood pulses of the LCX 201 temporarily increasing the pressure within the LCX 201 and thereby pushing on the vein wall 272.

Each of the sensors 224 and 225 can output one or more signals, the signals carried on one or more conductors of lead 226.

In some configurations, not all vein wall deflections detected by sensor 224 will be caused by systolic blood pulses of the LCX 201. For example, the motion of the heart, muscles, other body parts, and/or a change in position of a patient can cause vein wall deflections. Sensor 225 is positioned so that it likely senses the same vein wall deflections as sensor 224, except for vein wall deflections caused by systolic blood pulses of the LCX 201. The signal output by each of the sensors 224 and 225 can be compared to determine which vein wall deflections sensed by sensor 224 are caused by systolic blood pulses and which vein wall deflections are caused by something else (the later of which will likely be sensed by both sensors 224 and 225). The comparison of the signals can include subtracting one signal from another and/or correlating the timing of sensed deflection events, among other methods, to identify blood flow events and parameters of the LCX 201.

Various different types of sensors can be used to sense wall deflections. For example, a pressure transducer braced against a vein wall can sense vein wall deflections, the vein wall applying a force on the pressure transducer when the wall deflects. The pressure transducer can produce a voltage proportional to the force.

One or more strain gauges can also be used to measure wall deflections. A strain gauge can be positioned such that vein wall deflections impact the strain gauge, causing the stain gauge to flex. A stain gauge can also be coupled to a vein wall, such that a deflection of the vein wall causes changes in the dimension of the vein wall, flexing the strain gauge.

Pressure transducers and strain gauges can contain piezoelectric elements, such as crystals. Piezoelectric crystals can produce a voltage (electric signal) proportional to the force/deflection when deflected. Measuring a change in this electric signal can indicate a systolic blood pulse and/or blood pressure of a vessel adjacent to the vein in which the sensor resides.

Although pressure transducers and strain gauges using piezoelectric crystals are discussed herein, other types of pressure transducers and strain gauges are also contemplated, including capacitive and resistance based pressure transducers and strain gauges, among other sensors.

Optical sensors can also be used according to methods of the current invention to determine hemodynamic parameters. For example, the path of a beam of light traveling through an optical fiber can change as the optical fiber flexes. These changes in light beam path can be sensed by an optical sensor to detect a deflection of the optical fiber, the optical fiber deflection caused by deflection of a vein wall. Optical fibers can be modified such that deflections in specific areas along the length of the optical fiber cause more dramatic changes in a path of a light beam traveling through the optical fiber as compared to other portions of the optical fiber. A modified section of an optical fiber can be located along a portion of a vein wall, the portion of the vein wall adjacent to a vessel of interest. Because the especially sensitive portion of the fiber is located in a position where it is most likely to be deflected by vein wall movement caused by pulses within the vessel of interest, these events can be distinguished from deflections of other portions of the optical fiber.

Other sensors that could be used with devices and methods of the present invention to sense vein wall deflections include, but are not limited to, impedance sensors, inductive sensors, resistive sensors, capacitive sensors, single and multi-dimensional accelerometers, capacitive fluid pressure transducers, quartz based sensors, acoustic transducers, micro-electro-mechanical systems (MEMS), and ultrasonic transducers, among others. In embodiments where a reference sensor is used, the reference sensor may be the same type sensor or a different type sensor from the main sensor.

For example, an impedance sensor can be used to sense blood volume flowing within a vein, where a deflection of the vein wall affects blood volume and a corresponding impedance measurement.

In some embodiments, an accelerometer can be used to detect vessel wall deflections. For example, an accelerometer can be anchored by a structure such that the sensitive axis of the accelerometer is aligned with a predicted deflection axis of the vessel wall. In this orientation, the accelerometer is sensitive to movement along the axis of vein wall deflection but relatively insensitive to other motions (noise) not along the axis of vein wall deflection.

Sensors used to measure vessel wall deflections may be configured, for example, as a custom or off-the shelf devices. Sensors can be housed, coated, and/or encapsulated to optimize sensing, durability, and/or biocompatibility properties.

Not all systolic blood pulses in arteries will naturally cause wall deflections of an adjacent vein capable of being detected by a sensor. In such cases, it may be necessary for a sensor support member, or other structure, to bias the vein and/or artery such that they are in contact with one another and a systolic blood pulse in the artery will cause a detectable wall deflection of the vein. In some embodiments, the sensor support member may bias a first vessel so that it is in contact with a second vessel. In other embodiments, the sensor support member may bias a first vessel so that the wall of a second vessel is deflected.

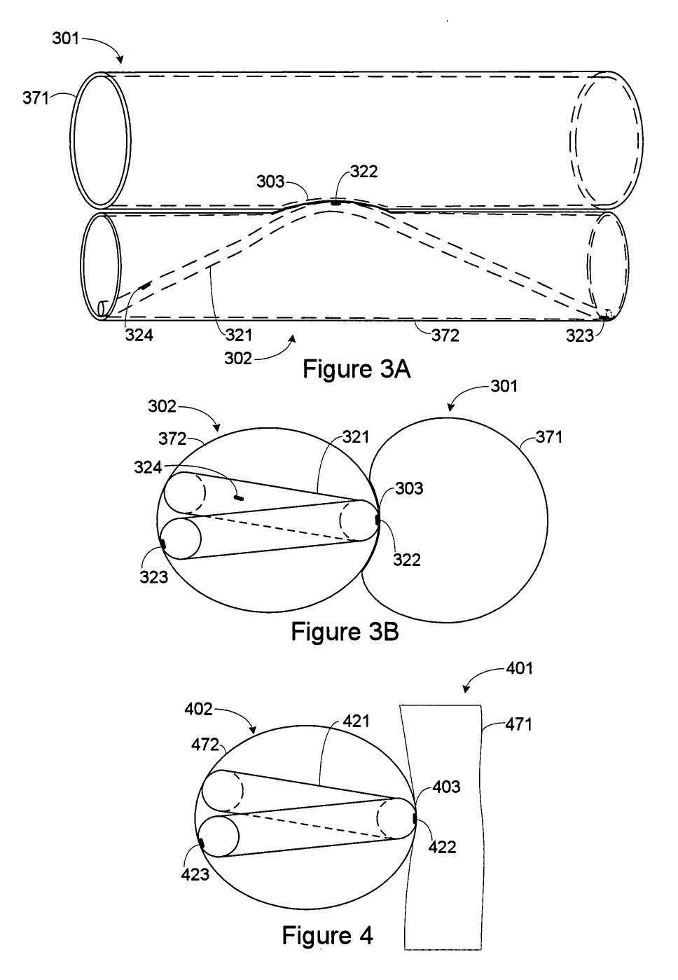

FIG. 3A illustrates a blood parameter sensing device used with two parallel vessels. The two parallel vessels are an artery 301 and a vein 302. A lead 321 is disposed within the vein 302. The vein 302 does not naturally contact the artery 301. However, the lead 321 is biased such that the lead 321 deflects a section of the vein wall 372. The vein wall deflection may cause the vein wall 372 to contact the artery 301 and cause a bulge 303 in the artery 301. In some situations, more reliable and/or more accurate sensor information may be obtained if the deflection of the vein wall 372 produces a bulge 303 in the artery wall 371. Systolic blood pulses occurring within the artery 301 will deflect the artery wall 371, causing the vein wall 372 to deflect as well. The vein wall deflection can be sensed by sensor 322.

The lead 321 can include electrodes (not shown) and may be coupled to an implantable medical device (not shown). The lead 321 can contain multiple conductors, including one or more conductors for each of the sensors 322, 323, and 324. Conductors can be used to power the sensors and/or facilitate signal transmission.

The lead 321 may be configured to exhibit mechanical spring properties to keep it anchored and sustain tension to maintain contact with the artery 301 or maintain the bulge 303 in the artery 301. Baroreflexes, physiological changes, pressure changes, and other factors can necessitate tension in the lead 321 to maintain contact. Metal, plastics, and/or other materials may be used to impart the mechanical spring properties of the lead 321. For example, NiTi alloy (Nitinol) may be used to impart a bend in the lead 321. A lead may also be pose-able so that it can be custom fit for a particular patient, location, and/or application.

The lead 321 may include optimal reference sensors 324 and 323. Sensor 323 is positioned such that, like sensor 322, it is in contact with the vein wall 372. Sensor 323 is not as likely to sense wall deflections caused by systolic blood pulses in the artery 301 as is sensor 322. Therefore, vein 302 wall deflection events sensed by sensor 323 can be used to discriminate between vein deflections caused by systolic blood pulses in the artery 301 and vein deflections caused by other things (the latter of which will likely be sensed by both sensors 323 and 324).

Sensor 324 can be used to sense blood pressure in the vein 302. Comparison of signals produced by sensors 322 and 324 can be used to discriminate between systolic blood pulse events and other events and/or provide information regarding relative blood pressures sensed in the vein 302 and artery 301.

FIG. 3B illustrates a blood parameter sensing device from a perspective cutaway looking into two parallel vessels 301, 302. The two parallel vessels 301, 302 can be the artery 301 and vein 302 illustrated in FIG. 3A. FIG. 3B illustrates how the biasing of the lead causes the lead 321 to impinge upon the vein 302 to form a bulge 303 in the artery 301. Sensor 322 is positioned to sense vein wall deflections caused by systolic blood pulses within the artery 301. Optional reference sensor 323 is positioned to sense vein wall deflections that are not caused by systolic blood pulses within the artery 301. Optional reference sensor 324 is positioned to sense the blood pressure within the vein 302.

FIG. 4 illustrates a blood parameter sensing device within a vein 402 which is orthogonal to an artery 401 in a cross over configuration. The vein 402 and artery 401 may not naturally be in contact with one another. However, the lead 421 within the vein 402 is biased such that the vein 402 is in contact with the artery 401. For example, the vein 402 may take on an oblong shape and impinge on the artery 401. One possible effect of the biasing of the lead 421 is the formation of a bulge 403 in the wall of the artery 401.

Sensor 422 is positioned against the wall 472 of the vein 402, proximate the point of contact between the artery 401 and vein 402, such that a systolic blood pulse within the artery 401 that causes a deflection of the artery wall 471 and a corresponding deflection of the vein wall 472. The deflection of the vein wall 472 is detectable by the sensor 422. The timing and/or magnitude of vein wall 472 deflections can be used to determine heart rate and blood pressure within the artery 401, among other hemodynamic parameters.

The lead 421 may also include optional reference sensor 423. Sensor 423 is positioned to sense vein wall 472 deflections that would not likely be caused by events occurring within the artery 401, as sensor 423 is located in a region where the vein wall 472 is proximate the artery wall 471. Pressure measurements and wall deflections sensed by the sensor 423 can be used to determine venous blood pressure, as well as assist in arterial blood pressure and heart rate determination by canceling out noise and unrelated events sensed by sensor 422.

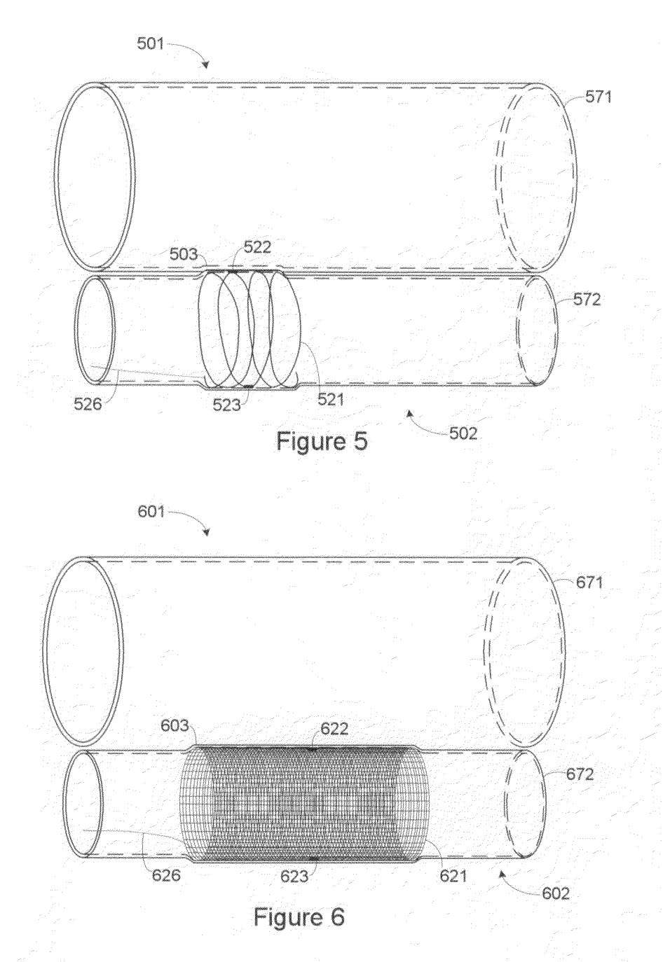

FIG. 5 illustrates a blood parameter sensing device within a vein 502, the vein 502 parallel with an artery 501. The vein 502 and the artery 501 may not naturally contact one another. However, a section of the vein 502 makes contact with a section of the artery 501. The contact between the vein 502 and the artery 501 may cause a bulge 503 in the artery 501. The vein 502 is caused to make contact with on the artery 501 because of the helix 521 located within the vein 502.

The helix 521 mechanically applies force to the inner vein 502 wall, which may cause the vein 502 to bulge along the length of the helix 521. To provide this mechanical force, the helix 521 may be under mechanical spring tension when constrained within the vein 502. Therefore, despite minor changes in vessel size (caused by vessel dilatation reflex, increased blood pressure, or by some other mechanism), the vein wall 572 will continue to make contact with the artery wall 571.

Sensor 522 is attached to the helix 521 and positioned to detect vein wall deflections, particularly wall deflections caused by a systolic blood pulse within the artery 501. Without the mechanical spring tension of the helix 521 forcing contact between the vein 502 and artery 501, the sensor 522 may not be able to sense events occurring in the artery 501, including systolic blood pulses. In some embodiments, sensing may be enhanced if the mechanical spring tension of the helix 521 causes a bulge 503 in the artery wall 571. As such, the helix 521, like other devices disclosed herein, allows sensor 522 to be ideally positioned to sense arterial wall deflections, arterial pressure, and arterial events by sensing vein wall deflections proximate the point of contact between the artery 501 and the vein 502.

Sensor 522 can generate one or more signals containing arterial hemodynamic parameter information and send the signals to circuitry using a conductor within or attached to the helix 521, such as lead 526. Conductors of lead 526 may also power the sensors 522, 523. Conductor 526 may also be integrated into, or attached to, the helix 521 to facilitate power and signal transmission.

Optional reference sensor 523 can also generate and transmits signals using the helix 521 and conductor 526. Sensor 523 is positioned to conduct pressure measurements and sense wall deflections, which can be used to determine venous blood pressure, as well as assist in arterial blood pressure and heart rate determination by canceling out noise and unrelated events sensed by sensor 522.

FIG. 6 illustrates a blood parameter sensing device within a vein 602, the vein 602 parallel with an artery 601. The vein 602 and the artery 601 may not naturally contact one another. However, a section of the vein 602 makes contact with a section of the artery 601, and may cause a bulge 603 in the artery 601. The vein 602 is caused to make contact with the artery 601 because of the stent 621 located within the vein 602.

The stent 621 applies mechanical force to the inner vein 602 wall, causing the vein 602 to expand along the length of the stent 621. Stent 621 can apply constant circumferential pressure outward when constrained within a vessel. Therefore, despite minor changes in vessel size, the vein wall 672 will continue to impinge upon the artery wall 671. The stent 621, as well as other components, anchor the sensors 622 and 623 against or proximate the vein wall 672, such that the sensors 622 and 623 can maintain the ability to sense wall movement despite vein or body movement, pulses, blood flow, and other factors which could otherwise cause sensors to lose contact with, and/or sensing capabilities of, the vein wall 672.

Sensors 622 and 623 can sense vein wall pressures and deflections, and send signals corresponding to pressure and/or deflection measurements using conductors within the lead 626.

The conductors of lead 626 may power the sensors 622, 623. Conductor 626 may also be integrated into, or attached to, the stent 621 to facilitate power and signal transmission.

The helix 521 of FIG. 5 and the stent 621 of FIG. 6, as well as other components disclosed herein, can be made from plastic, metal, and/or any other materials suitable for implantation. The helix 521 and stent 621 may be Nitinol or stainless steel, either uncoated or coated with a polymer and/or a drug.

Devices and methods to facilitate deployment of a stent within a vessel are known in the art. Similar devices and methods can also be used to deploy a helix, ring, or other structure within a vessel.

Imaging techniques can be used to identify adjacent vessels that could be used with devices and methods described herein, as well as aid in placement of structures and sensors within vessels so that a sensor in a vein can sense wall deflections of that vein corresponding to blood flow events in an adjacent vessel.

In some embodiments of the invention, acoustic sensing and/or imaging devices and techniques can be used to ensure that turbulent blood flow is not induced in the target vessel and/or the adjacent vessel by implantation of the devices referenced herein.

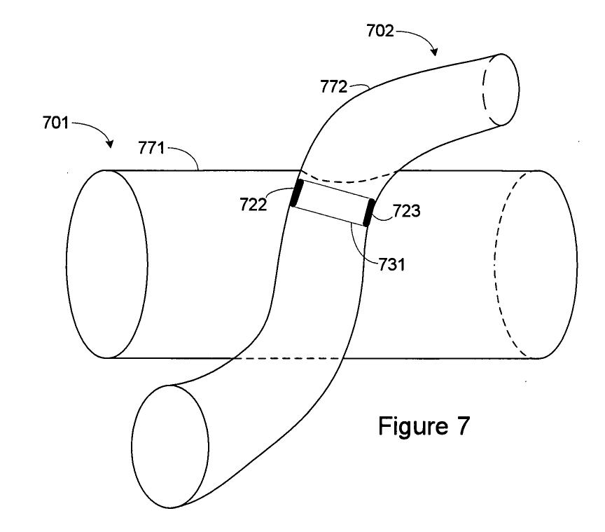

FIG. 7 illustrates a hemodynamic parameter sensing device within a vein 702, the vein 702 adjacent to an artery 701. Ultrasonic transducers 722 and 723 are each attached to inside walls of vein 702, the transducers 722 and 723 are supported by a flexible ring 731. The ultrasonic transducers 722 and 723 can be configured such that one of the ultrasonic transducers 722 and 723 sends ultrasonic pulses to the other of the ultrasonic transducers 722 and 723 to measure a distance between then ultrasonic transducers 722 and 723. By measuring the distance between the ultrasonic transducers 722 and 723, changes in dimension of the vein 702 can be detected. Detected changes in vein dimension can be associated with systolic blood pulses of the artery 701 causing the outer surface of the artery wall 771 to apply a force on the surface of the vein 702 wall, momentarily changing the vein 702 wall dimension, as detected by ultrasonic transducers 722 and 723.

Ultrasonic transducers 722 and 723 can be biased against, stitched, adhered, and/or integrated into the vessel wall (e.g., by tissue encapsulation) for anchoring.

Ultrasonic transducers 722 and 723 may use signal intensity, timing of sending and receiving pulses, and/or a Doppler shift effect to measuring vein dimension changes. Although two ultrasonic transducers are illustrated in FIG. 7, a single ultrasonic transducer can be used, the single ultrasonic transducer measuring the time of sending ultrasonic pulses and receiving a return echo pulse. Also, sonic or acoustic transducers may also be similarly used with the methods and devices discussed herein.

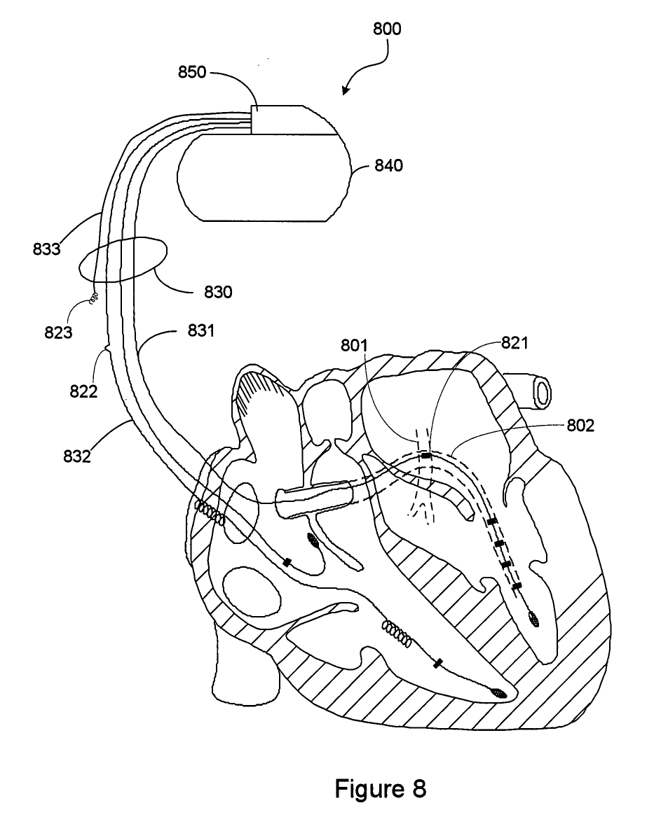

FIG. 8 illustrates a therapy device 800. The therapy device 800 illustrated in FIG. 8 employs circuitry capable of detecting and treating medical conditions, including bradycardia, tachyarrhythmia, and fibrillation, among others, as well as monitoring hemodynamic parameters. The therapy device 800 includes cardiac rhythm management (CRM) and monitoring circuitry enclosed within an implantable housing 840. The circuitry is electrically coupled to a lead system 830, the lead system 830 connected to the therapy device 800 via header 850.

Although a lead system 830 including intracardiac electrodes is illustrated in FIG. 8, various other types of lead/electrode systems may additionally or alternatively be deployed. For example, the lead/electrode system may comprise an epicardial lead/electrode system and/or leads and electrodes outside the heart and/or cardiac vasculature, such as a heart sock, an epicardial patch, and/or a subcutaneous system having electrodes implanted below the skin surface but outside the ribcage.

Portions of the intracardiac lead system 830 are inserted into the patient's heart. The lead system 830 includes cardiac pace/sense electrodes positioned in, on, or about one or more heart chambers for sensing electrical signals from the patient's heart and/or delivering electrical stimulation to the heart. The intracardiac sense/pace electrodes may be used to sense and/or pace one or more chambers of the heart, including the left ventricle, the right ventricle, the left atrium and/or the right atrium. The CRM circuitry controls the delivery of electrical stimulation pulses delivered via the electrodes. The electrical stimulation pulses may be used to ensure that the heart beats at a hemodynamically sufficient rate, may be used to improve the synchrony of the heart beats, may be used to increase the strength of the heart beats, and/or may be used for other therapeutic purposes to support cardiac function consistent with a prescribed therapy.

The lead system 830 of the therapy device 800 includes intracardiac lead 831, a portion of the intracardiac lead 831 located within vein 802. The vein 802 crosses over the LCX coronary artery 801, such that the vein 802 and artery 801 are proximate one another. Intracardiac lead 831 may contain any of the structures discussed herein for forcing mechanical contact between a portion of the vein 802 and the artery 801 at the crossover point such that systolic blood pulses in the artery 801 exert a force on the artery 801 wall to deflect the vein 802 wall.

A sensor 821 attached to the lead and is configured to detect vein 802 wall deflections corresponding to the systolic blood pulses in the artery 801. The sensor 821 can transmit one or more signals containing deflection information through the intracardiac lead 831 to circuitry of the implantable housing 840.

Leads 832 and 833 may be used each alone, or together, with the other leads of the lead system 830. Intracardiac lead 832 includes a bias point 822. One or more sensors can be placed on and/or around the bias point 822 to measure pressure and/or detect vessel wall deflections. The bias point 822 can facilitate the sensing of a vein wall deflection corresponding to a systolic blood pulse of an adjacent artery, as discussed herein.

Lead 833 does not include an electrode, but does include a helix 823. The helix 823 can be deployed within a vessel and used with a sensor to monitor the hemodynamic status of an adjacent vessel. As illustrated, lead 832 and helix 823 are not located in the heart vasculature, but can be used in vessels elsewhere in the body. For example, helix 823 may be placed within a vein in the neck to the monitor hemodynamic status of a patient, the vein adjacent an artery.

Communications circuitry is disposed within the housing 840 for facilitating communication between the CRM circuitry and a patient-external device, such as an external programmer or advanced patient management (APM) system. The therapy device 800 may also include sensors, including vessel wall deflection sensors, and appropriate circuitry for sensing a patient's metabolic need and hemodynamic status and adjusting the pacing pulses delivered to the heart to accommodate the patient's metabolic need if the sensed hemodynamic status is not appropriate for the patient's metabolic need.

In some implementations, an APM system may be used to perform some of the processes discussed herein, including signal processing, comparing signals and sensed hemodynamic events, storing data, and directing therapy, among others. Methods, structures, and/or techniques described herein, may incorporate various APM related methodologies, including features described in one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference in each of their respective entireties. These and other therapy methodologies can be directed by the blood flow parameters measurements discussed herein.

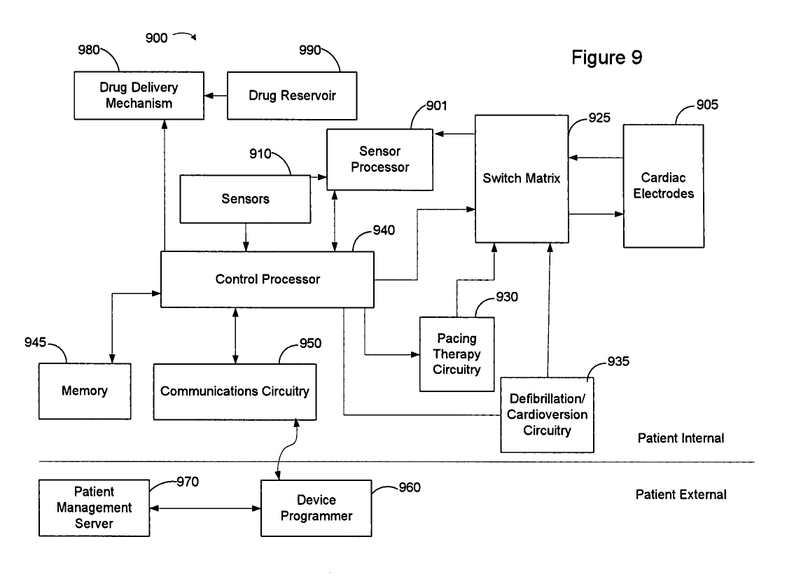

FIG. 9 is a block diagram of a CRM device 900 that may incorporate sensors for detecting vessel wall deflections to allow monitoring various hemodynamic parameters of a patient. The CRM device 900 may include pacing therapy circuitry 930 that delivers pacing pulses to a heart. The CRM device 900 may include defibrillation/cardioversion circuitry 935 configured to deliver high energy defibrillation or cardioversion stimulation to the heart for terminating dangerous tachyarrhythmias.

The electrical therapy pulses can be delivered via multiple cardiac electrodes 905 (electrode combinations) disposed at multiple locations within a heart and/or elsewhere in the patient's body. The electrodes 905 are coupled to switch matrix 925 circuitry used to selectively couple electrodes 905 of various electrodes 905 to sensor processor 901 and/or other components of the CRM device 900. The sensor processor 901 is also coupled to one or more sensors 910, which can be any of the sensors discussed herein or other sensors.

Sensor processor 901 is configured to receive information gathered via the cardiac electrodes 905 and sensors 910. The sensor processor 901 can perform various functions, including comparing EGM data to detected hemodynamic events, such as systolic blood pulses corresponding to ventricular contractions. Comparison of EGM data with detected hemodynamic data can confirm EGM events and/or sensed hemodynamic events. For example, a heart rate determined using sensed vessel wall deflections can be used to confirm a heart rate sensed via EGM or ECG, and visa versa. Other parameter values independently determined by EGM or ECG and vessel wall deflection sensors can be combined, such as averaging a heart rate value determined from an ECG or EGM with a heart rate value determined by vessel wall deflection sensors.

The control processor 940 can use hemodynamic parameter information received from sensors 910 to diagnose cardiac conditions and/or direct therapy.

Parameter values, responsive to vessel wall deflections, can indicate a state of blood flow of a vessel, such as an artery. In such a way, a parameter value indicative of blood flow in an artery can be used by circuitry to initiate or postpone therapy. For example, if sensed ECG signals erroneously indicate a need for anti-tachycardia pacing (ATP) therapy (usually a high energy shock), but the parameter value indicates sufficient blood flow in an artery, ATP therapy can be postponed or delayed. Therefore, the present invention can minimize the delivery of unnecessary ATP therapy, which can otherwise be uncomfortable for patients and consume energy resources.

Bradycardia, tachyarrhythmia, or fibrillation conditions detected using sensed vein wall deflections, as discussed herein, could be treated by electrical stimulation delivered using the pacing therapy circuitry 930 or defibrillation/cardioversion circuitry 935 and cardiac electrodes 905. These conditions can be detected and/or confirmed using the blood flow parameters discussed herein based on vessel wall deflections.

The sensor and/or circuitry of the CRM 900 may have signal conditioning circuitry such as high pass and/or low pass filters. For example, a band pass filter may be used to focus on heart rate (e.g., passing signal frequencies corresponding to the heart rate range of a typical human).

Physiologic conditions can be treated using various drugs stored in drug reservoir 990 and delivered using a drug delivery mechanism 980. Drug delivery mechanism 980 could be any drug delivery mechanism known in the art for delivering drugs, including a valve, bellows, pressurization, electrophoresis, and sonic phoresis, among others. Drug delivery devices that are primarily external are also contemplated within the scope of the present invention, including drug delivery devices that employ intravenous delivery of drugs using a needle or skin port.

Drug delivery mechanism 980 can deliver one or more therapeutic drugs in response to detection of physiologic conditions sensed using vein wall deflection detection methods and devices disclosed herein. For example, sensor processor 901, receiving one or more signals from sensors 910, may detect a hypertension condition using the devices and methods disclosed herein. In response to the hypertension diagnosis (chronic or acute), the control processor 940 can direct the drug delivery mechanism 980 to deliver a therapeutic drug to address the hypertension condition. Therapeutic drugs can include, but are not limited to, ACE inhibitors, angiotensin II receptor antagonists, alpha blockers, beta blockers, calcium channel blockers, and/or diuretics.

In some embodiments, the sensor processor 901 can determine the patient's status based on the vein wall deflection methods described herein. The control processor 940 may initiate notification of the patient and/or the patient's physician regarding the patient's status. If a change in status occurs, the control processor 940 may initiate an alert which may be sent to the patient and/or physician via a mobile communication device, device programmer, and/or APM server. In some embodiments, the control processor 940 or the APM server may evaluate the patient status data and recommend modifications to the patient's therapy, such as a change in the patient's pacing parameters or medication.

Sensor processor 901 can continue to monitor hemodynamic parameters using the sensors 901 to evaluate the therapy effectiveness. Based on changes in the patient's condition and therapy effectiveness, the control processor 940 can direct the drug delivery mechanism 980 or pacing therapy circuitry 930 to automatically modify or stop the drug and/or pacing therapy. Alternatively, or additionally, the control processor can communicate with the patient's physician to make recommendations regarding changes to the patient's medication or other prescribed therapy. In this way, devices and methods of the present invention can participate in closed-loop therapy systems.

A CRM device 900 typically includes a battery power supply (not shown) and communications circuitry 950 for communicating with an external device programmer 960 or other patient-external device. Information, such as data, hemodynamic status, and/or program instructions, and the like, can be transferred between the device programmer 960 and patient management server 970, CRM device 900 and the device programmer 960, and/or between the CRM device 900 and the patient management server 970 and/or other external system. In some embodiments, the sensor processor 901 may be a component of the device programmer 960, patient management server 970, or other patient external system.

The CRM device 900 also includes a memory 945 for storing program instructions and/or data, accessed by and through the control processor 940. In various configurations, the memory 945 may be used to store information related to hemodynamic status, vessel wall deflections, parameters, measured values, program instructions, data, and the like.

The circuitry represented in FIG. 9 can be used to perform the various methodologies and techniques discussed herein. Memory 945 can be a computer readable medium encoded with a computer program, software, computer executable instructions, instructions capable of being executed by a computer, etc, to be executed by circuitry, such as control processor 940 and/or sensor processor 901. For example, memory 945 can be a computer readable medium storing a computer program, execution of the computer program by control processor 940 causing reception of one or more signals from sensors 910, measurement of the signals, calculation using one or more algorithms, and outputting of a parameter, such as blood pressure or heart rate, according to the various methods and techniques made known or referenced by the present disclosure. In similar ways, the other methods and techniques discussed herein can be performed using the circuitry represented in FIG. 9.

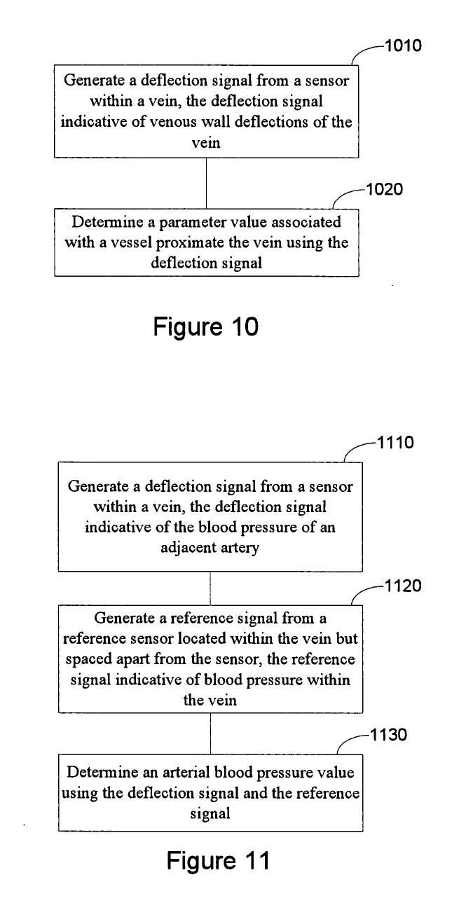

The flowchart of FIG. 10 illustrates a process for monitoring the hemodynamic status of a vessel from an adjacent vessel. A deflection signal is generated 1010 by a sensor within a vein, the deflection signal indicative of venous wall deflections of the vein. The process can use any of the sensors, alone or in combination, disclosed herein or other sensors capable of detecting vessel deflections.

The process of FIG. 10 further includes determining 1020 a parameter value associated with a vessel proximate the vein using the deflection signal. The parameter value could be any of the parameters disclosed herein or other parameters that can be determined using the deflection signal. The vessel could be an artery, lymph vessel, vein, or other fluid carrying vessel of the body adjacent to another vessel.

The flowchart of FIG. 11 illustrates a process for monitoring arterial blood pressure from a vein adjacent an artery. A deflection signal is generated by a sensor within a vein, the deflection signal indicative of the blood pressure of an adjacent artery. The sensor can measure blood pressure from an adjacent artery by measuring a force applied to the sensor by the inside surface of a wall of the vein in which the sensor resides, where the outside surface of the vein wall is in contact with the outside surface of a wall of the artery.

The process of FIG. 11 further includes generating 1120 a reference signal from a reference sensor located within the vein but spaced apart from the sensor, the reference signal indicative of blood pressure within the vein. The reference sensor may be in contact with the wall of the vein, or it may not be in contact with a vein wall.

The process of FIG. 11 further includes using 1130 the reference signal to increase the signal to noise ratio of the deflection signal to determine an arterial blood pressure value. For example, in some embodiments, the reference signal may be subtracted from the deflection signal. The signal after subtraction may be used to determine the arterial pressure value. For example, the signal after subtraction may be compared to a calibration standard to determine the arterial pressure values. A calibration standard may be determined for any of the signals discussed here by measuring arterial blood pressure using any of the methods disclosed herein and comparing that value to the known blood pressure (determined using traditional methods) multiple times across multiple arterial blood pressures. From these measurements a proportional relationship between the signal(s) and blood pressure can be established, such that a particular signal value (e.g., measured in volts) will correspond to a blood pressure value (e.g., measured in mm Hg). Likewise, a particular change in a particular signal (e.g. measured in volts) can correspond to a change in blood pressure (e.g. measured in mm Hg) and can correspond to a heart beat or a change in cardiac status.

Various algorithms can be used with the signals and parameters discussed herein. Such algorithms include, but are not limited to, averaging, ensemble averaging, parameter extractions, morphology mapping, dP/dt, Pk/Avg, diurnal cycle identification, and variable delay differencing. For example, rates of pressure variation can be evaluated and mapped back to equivalent cardiac dP/dt values. These can be used for diagnostic aids or for the modulation of device therapy.

The components, functionality, and structural configurations depicted herein are intended to provide an understanding of various features and combination of features that may be incorporated in an implantable pacemaker/defibrillator or other implantable medical device. It is understood that a wide variety of monitoring and/or therapy device configurations are contemplated, ranging from relatively sophisticated to relatively simple designs. As such, particular device configurations may include particular features as described herein, while other such device configurations may exclude particular features described herein.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. An implantable transvascular pressure sensing device, comprising:
at least one deflection sensor configured to output a deflection signal indicative of vascular wall deflections; and
an implantable sensor support member attached to the deflection sensor, the sensor support member configured to facilitate contact between the deflection sensor and a first vessel wall at a portion of a first vessel proximate a second vessel, wherein the portion of the first vessel wall is adjacent to the second vessel such that wall deflections of the second vessel deflect the portion of the first vessel wall.

2. The device of claim 1, further comprising circuitry configured to determine the pressure within the second vessel based on the signal indicative of first vessel wall deflections.

3. The device of claim 2, further comprising at least one reference sensor configured to be positionally maintained within the first vessel and out of contact with the portion of the first vessel, the reference sensor further configured to output a reference signal, wherein the circuitry is further configured to determine the pressure in the second vessel using the deflection signal indicative of first vessel wall deflections and the reference signal.

4. The device of claim 1, further comprising circuitry configured determine a heart rate based on the deflection signal.

5. The device of claim 1, wherein the first vessel is a coronary vein and the second vessel is a left circumflex coronary artery.

6. The device of claim 1, wherein the sensor support member comprises a stent.

7. The device of claim 1, wherein the sensor support member comprises a coil.

8. The device of claim 1, wherein the sensor support member comprises a helix.

9. The device of claim 1, wherein the support member is configured to facilitate and maintain a point of contact between the first vessel and the second vessel, the point of contact proximate one of the deflection sensor.

10. The device of claim 1, wherein the sensor support member comprises a lead.

11. The device of claim 1, wherein the deflection sensor comprises an optical fiber sensor.

12. The device of claim 1, wherein the deflection sensor comprises one or more accelerometers.

13. The device of claim 1, wherein the deflection sensor comprises one or more pressure transducers.

14. The device of claim 1, wherein the deflection sensor comprises one or more strain gauges.

15. The device of claim 1, wherein the deflection sensor comprises one or more electrodes configured to sense impedance of the first vessel.

16. A method of measuring a parameter of a vessel, the method comprising:
generating a deflection signal from a sensor within a vein, the deflection signal indicative of venous wall deflections of the vein; and
determining a parameter value associated with a vessel contacting the vein using the deflection signal.

17. The method of claim 16, wherein the parameter is pressure.

18. The method of claim 16, further comprising:
generating a reference signal from a reference sensor located within the vein but spaced apart from the sensor, the reference signal indicative of venous wall deflection of the vein; and
determining the parameter value using the deflection signal and the reference signal.

19. The method of claim 16, wherein the vessel is an artery.

20. The method of claim 16, wherein the vein is a coronary vein and the vessel is a left circumflex coronary artery.

21. The method of claim 16, further comprising supporting the sensor using one or more of a stent, a helix, and an annular support member within the vein such that the sensor maintains contact with a wall of the vein, the wall of the vein in contact with the wall of the vessel.

22. The method of claim 16, further comprising placing a vein biasing structure within the vein, the vein biasing structure biasing the vein such that an outer surface of the vein maintains contact with an outer surface of the vessel at a point of contact proximate the sensor.

23. The method of claim 16, further comprising extending an anti-tachycardia pacing interval based on the parameter indicating a state of blood flow in the vessel.

24. A system for measuring a parameter of a vessel, the method comprising:
means for generating a deflection signal from a sensor within a vein, the deflection signal indicative of venous wall deflections of the vein; and
means for determining a parameter value associated with a vessel contacting the vein using the deflection signal.

25. The system of claim 24, further comprising:
means for generating a reference signal from a reference sensor located within the vein but spaced apart from the sensor, the reference signal indicative of venous wall deflection of the vein; and
means for determining the parameter value using the deflection signal and the reference signal.
